(19) United States
(12) Patent Application Publication
(10) Pub. No.: US 2011/0046630 A1
(43) Pub. Date: Feb. 24, 2011

(54) METHOD AND KIT FOR INTRA OSSEOUS NAVIGATION AND AUGMENTATION OF BONE

(76) Inventor: Kieran P. Murphy, Toronto (CA)

Correspondence Address: OSTROLENK FABER GERB & SOFFEN, 1180 AVENUE OF THE AMERICAS, NEW YORK, NY, 100368403, US

(21) Appl. No.: 12/449,425

(22) PCT Filed: Mar. 12, 2007
(86) PCT No.: PCT/US2007/063766

§ 371 Date: Nov. 10, 2010

(51) Int. Cl. A61B 17/58; A61B 17/56; A61M 5/00

(52) U.S. Cl. 606/94; 606/86 R; 604/272

(57) ABSTRACT

A method and kit for navigation and augmentation of bone is provided. In an embodiment, the method comprises creating a passageway in an end of a long bone and inserting a cannula or the like into the passageway. The cannula, or other instrument inserted through the cannula, can be used to break trabecula within the long bone. The cannula can also be used to inject a bone cement into the long bone. Where a biologically active bone cement is used, the method can also comprise adminstering a medication, such as PTH, to stimulate integration of the bone cement with the long bone.

### FIELD

The present application relates generally to treatment of bone fractures and more specifically relates to a method and kit for intra osseous navigation and augmentation of bone, such as bone fractures or weakened bone.

### BACKGROUND

Fractures of the radius, the principal bone of the forearm, occur with increasing frequency with age. They usually occur secondary to fall on an outstretched arm. This mechanism was initially described by Abraham Colles in 1870. He was a surgeon in The Royal College of Surgeons in Ireland. An alternate mechanism was the fall on the flexed wrist which is called a Smith fracture. He was a surgeon at Trinity College, Dublin. Since that time, the method of repair has been closed reduction, i.e., general anesthesia or deep conscious sedation with manipulation of the bone fragments to realign them.

Radial fractures therefore have a long history in medicine. In osteoporotic women, three principal fractures occur. These arc fractures of the femoral neck, thoracic and lumbar spine compression fractures and radial fractures. Once a patient reaches a level of osteoporososis or bone reabsorption secondary to age or the use of a medication such as a steroid for immunosuppression perhaps in transplantation, the incidence of fractures increase. It is known that if you have one fracture of a vertebral body for example, there is a 10 percent per year risk of a second fracture. The traditional methods of treating these fractures include the use oral medications to increase bone density. In women with profoundly weakened bone, this may not work as their bone is unable to react to these medications. Many of these bone medications are essentially bone poisons that decrease bone turnover and therefore decrease the rate or bone reabsorption or slow loss of bone.

During the last 20 years, there has been an explosion of image-guided therapy development both on the guidance side with extra machinery and on the device side with catheters, wires and needles for navigation. At the same time, there has been a growth in the array of bone cements which are available for surgical implantation into bone. There are very, very few of these cements that can be injected under image guidance due to their chalky, physical properties and the need to apply pressures to these cements to make them injectable through narrow devices which results in dewetting of the cement and therefore increasing its chalkiness or resistance to injection with an overall loss of injectable physical properties.

Navigation in vertebra has been published discussed previously in “Multilevel Vertebroplasty Via A Single Pedicular Approach Using Curved 13-Gauge Needle: technical note”, Can. Assoc. Radiol. J. 2002; 53(5):293-5., Kieran J. Murphy et. al. (“Murphy”) Navigation in the femoral neck has been performed in research activities. However, the technique described in Murphy is not suitable for long bone navigation. The metal needle use in that paper has a sharp tip that is necessary to penetrate the near solid nature of Cortical bone or sclerotic trabecular bone. The tip is extremely sharp in order to penetrate cortical and dense bone. The curvature of the needle is fixed at the time of its manufacture. It is also known to repair or prevent fracture in patients with high risk involve the use of orthopedic metal implants and bone cements that are injected on their own without the adjunctive use of oral medications to promote their integration. Some of these bone cements are not integratable, and are poorly adhesive to bone, particularly those based in polymethylmethacrylate (PMMA).

### SUMMARY

Various methods, kits and apparatuses for intra osseous navigation and augmentation of bone are provided., Various aspects and embodiments include, 1) the application of image-guided therapy technique to long-bone augmentation; 2) using image guidance, needles, wires and catheters usually used in arterial access; and 3) injectable cements that are then stimulated by the addition of an oral medication or subcutaneous medication to promote their integration.

Techniques described herein can, amongst other things, allow for the prophylactic augmentation of unfractured bone in particular. Fracture of one radius is associated with a 10 to 20 percent chance of fracture of the other radius. In order to obviate the pain, suffering and loss of mobility associated with bilateral fracture, the prophylactic augmentation of the unfractured radius at the time of the fracture of the first radius can be performed using the teachings herein.

Various methods are proposed. The methods can be applied after reduction of a fracture or, in a patient at high risk for fracture, perhaps because of a preceding contra lateral fracture. Thus, the methods can be applied to augment prophylactically an unfractured bone.

Bones, specifically, but not exclusively, that can be treated using the teachings herein include bones such as the distal radius, the proximal femur, the distal and proximal tibia, the proximal femur and the iliac and pelvic bones. The teachings also contemplate the injection of biologically active bone cements and the use of additional medications to assist in the integration of the bone cements.

The current disclosure also contemplates the use of a nylon, plastic or reinforced polymer tubes with shaped tips, to deliver bone cement strategically within the predominantly hollow space of long bones prone to fracture or already fractured. Such a polymer tube is configured for intra osseous navigation such that the tube (i.e. catheter) is able to resist compression from longitudinal loading that exceeds that of a vascular catheter. The tube can be a compressible polymer coated braided coil with a shapeable tip. It can be guided to its target location in the bone by a combination of its shape and the shape of the very stiff wire that fits through the coil (which can also be referred to as a catheter). The coil can be a polymer coated coil.

An aspect provides a method for augmentation of a bone comprising:
piercing an end of the bone with a trocar until a distal tip of the trocar reaches a target area within the bone; the trocar comprising a stylet and a first cannula;
removing a stylet from the first cannula leaving a passageway through the first cannula into an interior of the bone terminating at the target area;
inserting a guide needle into the first cannula;
passing a larger needle over the guide needle and into the bone; the larger needle being worked so as to increase a size of the passageway until the passageway is of a sufficient diameter to receive a shorter cannula wider than the first cannula; and
inserting the shorter cannula into the passageway.

The method can comprise inserting an instrument into the passageway through the shorter cannula to break trabecular septations.

The method of claim can further comprise
introducing a stiff guidewire into the shorter cannula;
passing a hollow curved needle over the stiff guide wire; the hollow curved needle having a curved distal tip; the curve being positioned within the target area;
rotating a hub of the hollow curved needle such that the curve breaks residual trabecular septations.

The method can further comprise: passing a stiff guide wire into the passageway to break residual trabecular septations.

The stiff guide wire is an Amplatz Super Stiff or a Rosen wire.

The method can further comprise passing a short flexible tube with metal braid into the passageway.

The short flexible tube can be inserted in such a manner as to further break residual trabecular septations.

The short flexible tube can further comprises a fitting for attaching a syringe. The syringe can be for delivering bone cement.

The method can further comprise injecting a bone cement through the flexible tube and into the bone.

The method can further comprise the step of injecting a bone cement into the target area. The bone cement can be a biologically active bone cement.

The method can further comprise administering a medication to stimulate the bone cement to integrate with the bone. The medication can be parathyroid hormone (“PTH”).

The bone can be any long bone, such as the radius or the humerus, the distal radius, the proximal femur, the distal and proximal tibia, the proximal femur and the iliac and pelvic bones.

The method can be performed after the bone has been fractured and then set, or it can be performed prophylactic.

The bone can be a vertebral body. The vertebral body can be between T8 and L2.

The method can further comprise performing the method under image guidance.

Another aspect provides a method for augmentation of a bone comprising:
creating a passageway in an end of the bone; and
inserting an object into the passageway to break residual trabecula within the bone.

The method can further comprise injecting a biologically active bone cement into the passageway.

The method can further comprising administering parathyroid hormone (“PTH”) to stimulate the bone cement to integrate with the bone.

Another aspect provides a kit of parts for augmentation of bone comprising a trocar comprising a first cannula and a removable stylet. A contiguous tip for piercing through an end of the bone is formed when the stylet is assembled with the first cannula. The kit also includes a guide needle for passing through the first cannula into the end of the bone when the stylet is removed from the first cannula. The kit also includes a hollow larger needle having a gauge larger than the guide needle. The larger needle is for passing over the guide needle when the first cannula is removed from the end of the bone and the guide needle is left within the end of the bone. The hollow larger needle is configured for breaking trabecula within the end of the bone to create a passageway of a predefined diameter.

The kit can further comprise a short cannula having a gauge larger than the hollow larger needle and a gauge less than or equal to the predefined diameter, the cannula presenting a passageway into the bone of the predefined diameter.

The kit can further comprise a stiff guide wire for insertion into the short cannula for breaking residual trabecular septations within the bone. The stiff guide wire can be one of an Amplatz or Rosen wire, or cross wire.

The kit can further comprise a curved needle. The curved needle has a straight portion and a curved portion at a distal end. The curved portion can be configured to have a diameter in the range about one mm to about one cm. The curve portion is configured for breaking residual trabecular septations within the bone when the curved needle is rotated within the bone. The curve can be configured to have a diameter in the range of about five mm.

The kit can further comprise a stiff guide wire for insertion into the hollow larger needle and for further optionally breaking the residual trabecula.

The kit can further comprise a metal braided tube and passing over the guide wire once the larger needle is removed. The metal tube is for further optionally breaking the residual trabecula.

### BRIEF DESCRIPTION OF THE DRAWINGS

FIG. 1 is a skeletal representation of the arm.

FIG. 2 shows the radius of FIG. 1 in greater detail.

FIG. 3 shows a kit of apparatuses for use in bone navigation and augmentation in accordance with an embodiment.

FIG. 4 shows the wrist of the arm and one suitable point for entry into the radius using the trocar of FIG. 3.

FIG. 5 shows the radius of FIG. 2 using the trocar.

FIG. 6 shows further use of various apparatuses from the kit of FIG. 3 to navigate the radius.

FIG. 7 shows further use of various apparatuses from the kit of FIG. 3 to navigate the radius.

FIG. 8 shows further use of various apparatuses from the kit of FIG. 3 to navigate the radius.

FIG. 9 shows further use of various apparatuses from the kit of FIG. 3 to navigate the radius.

FIG. 10 shows further use of various apparatuses from the kit of FIG. 3 to navigate the radius.

FIG. 11 shows further use of various apparatuses from the kit of FIG. 3 to navigate the radius.

FIG. 12 shows further use of various apparatuses from the kit of FIG. 3 to navigate the radius.

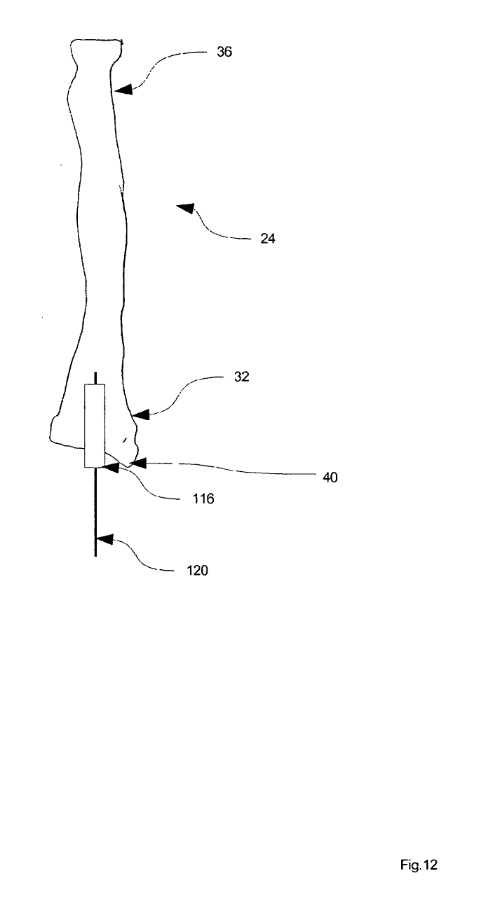

FIG. 13 shows further use of various apparatuses from the kit of FIG. 3 to navigate the radius.

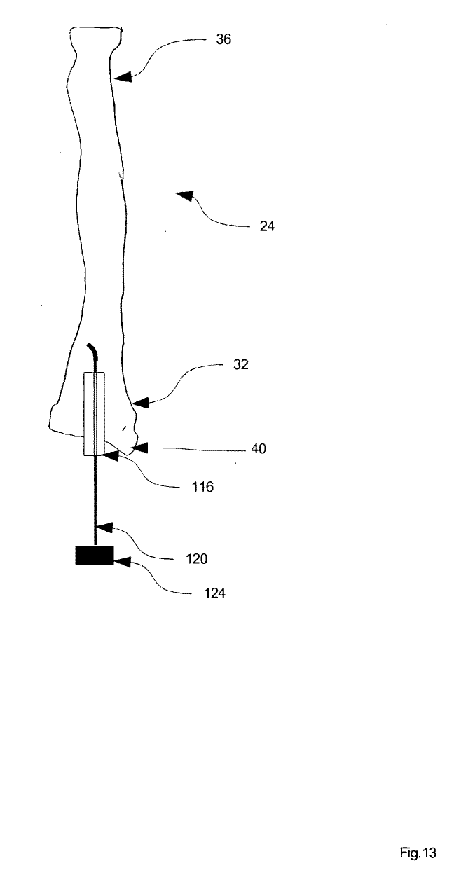

FIG. 14 shows optional further apparatuses that can be included in the kit of FIG. 3.

FIG. 15 shows use of the optional further apparatuses shown in FIG. 14.

FIG. 16 shows further use of the optional further apparatuses shown in FIG. 14.

FIG. 17 shows use of the optional further apparatuses shown in FIG. 14, including a syringe.

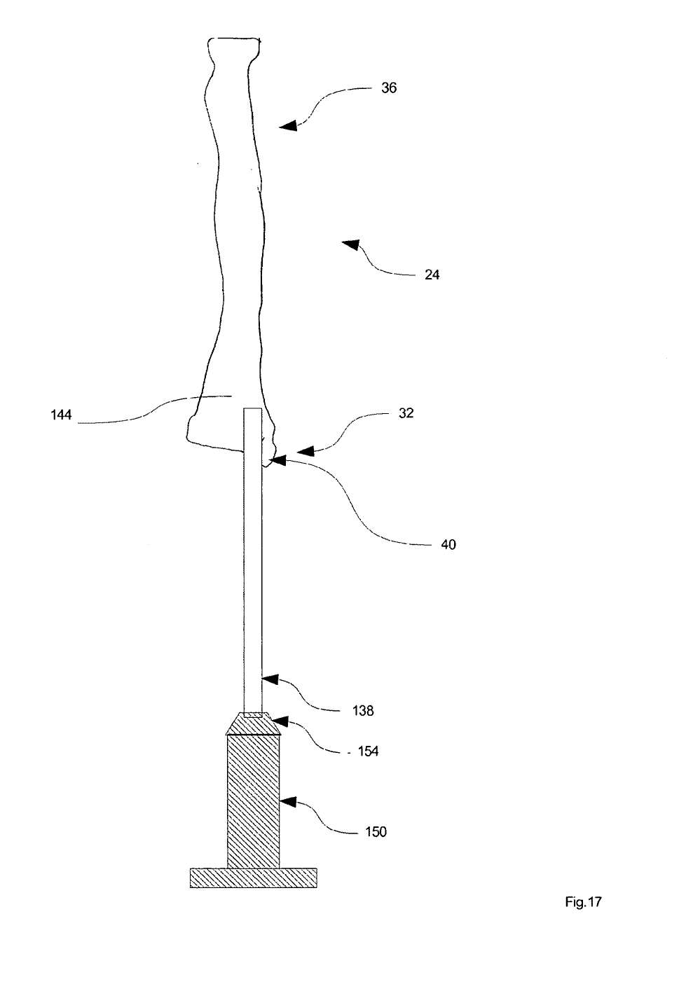

FIG. 18 shows another exemplary trajectory for entry into the radius shown in FIG. 2.

### DETAILED DESCRIPTION OF THE EMBODIMENTS

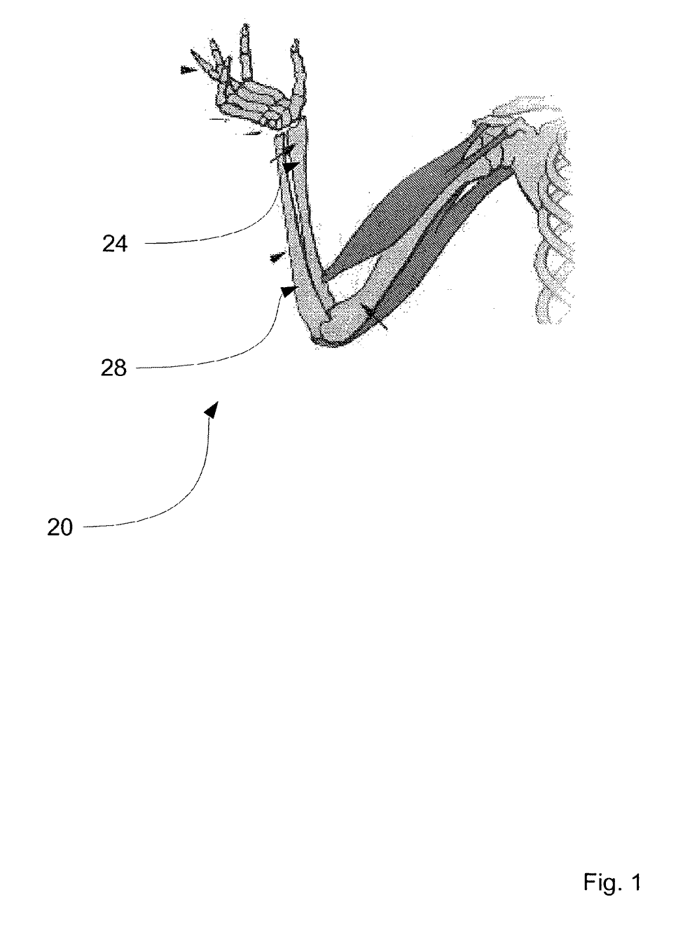

Referring now to FIG. 1, a skeletal representation of a human forearm is indicated generally at 20. As understood by those of skill art, forearm 20 includes a first long bone commonly referred to the radius indicated at 24. Forearm 20 also includes a second long bone commonly referred to as the ulna, and indicated at 28.

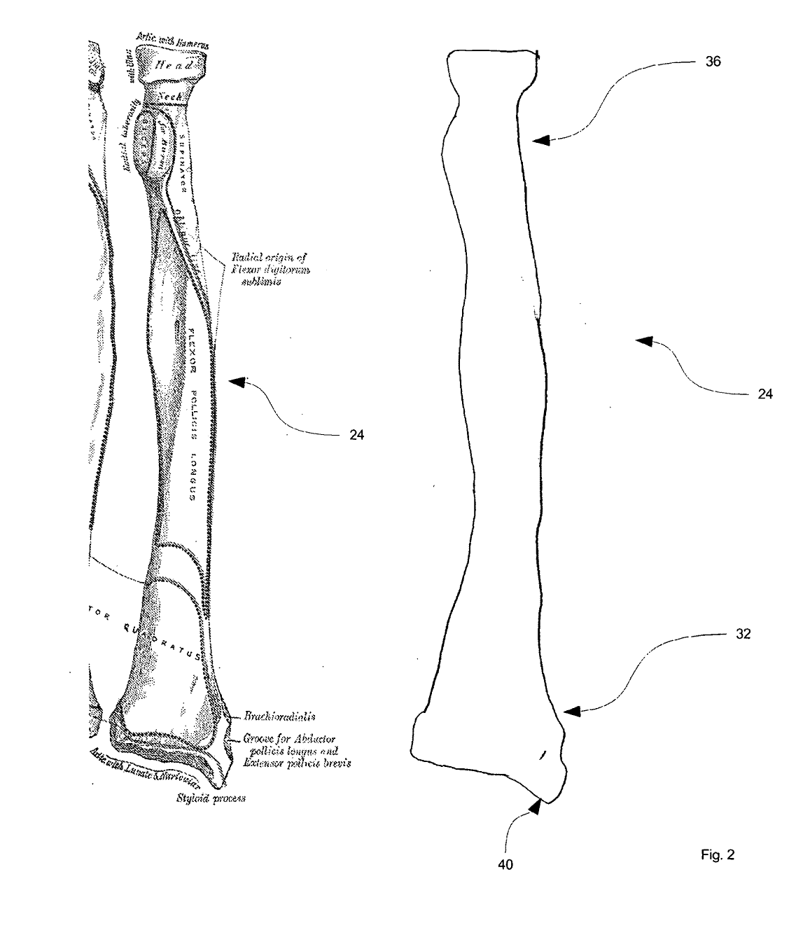

Radius 24 is shown in greater detail in FIG. 2. Of note, in FIG. 2 radius 24 is characterized by the distal radius 32, located distal end of radius 24 where radius 24 defines part of the wrist joint, and by the proximal radius 36 located at the proximal end of radius 24 where radius 24 defines part the elbow joint. As shown in FIG. 2, radius 24 is further characterized by the styloid process 40.

In one embodiment, a method for augmentation of a bone is provided. (In the present embodiment, the method is applied to the radius 24, but it should be understood that the method can be applied to other bones, including other long bones, such as, for example, the femur and the tibia.) The bone augmentation method for the present embodiment can be effected after reduction of a fracture of radius 24, whereby a fractured radius 24 has been properly “set” so that the fractured portions of radius 24 are oriented in a manner that will permit them to heal. The method can also be employed, prophylactically, in a patient at high risk for fracture, perhaps based on a patient having already injured the radius 24 on one arm, and therefore at risk of injuring the radius 24 on the other arm. The method can thus be applied to augment prophylactically an unfractured bone.

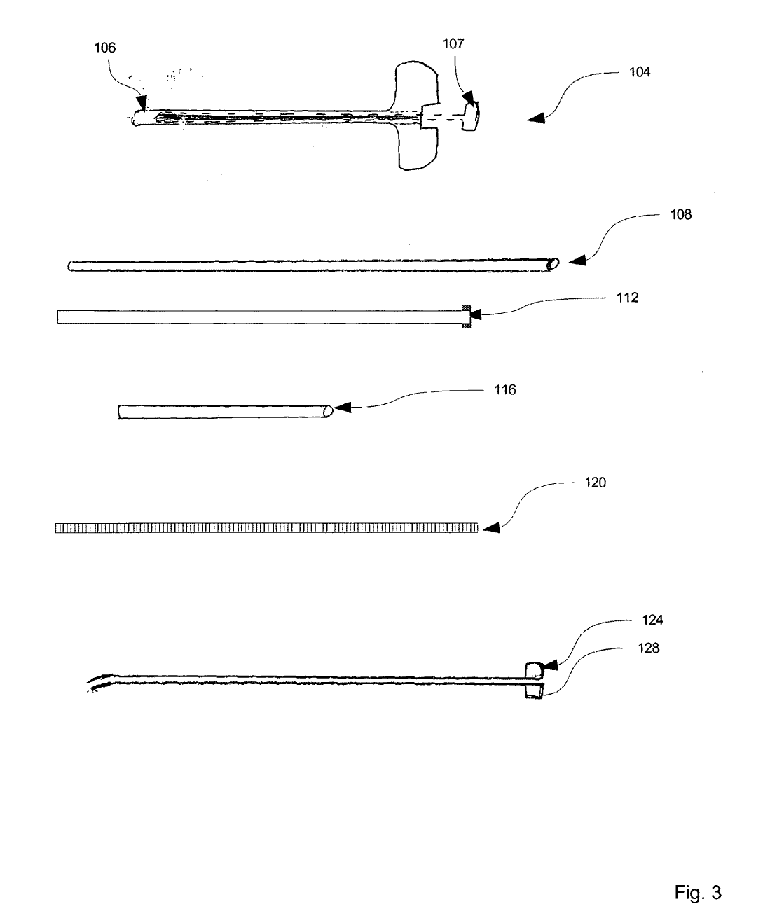

The method can be performed using a kit of apparatuses. An example of such a kit is shown in FIG. 3 and indicated at 100. Kit 100 comprises a trocar 104, which itself comprises a hollow cannula 106 and a stylet 107 that is received through cannula 106. When assembled cannula 106 and the tip of stylet 107 form a contiguous tip for piercing through tissue. Once piercing is complete, stylet 107 can be removed to present a hollow channel within cannula 106. Kit 100 also comprises a guide needle 108. The guide needle 108 can be of any desired gauge, such as an eight Gauge needle or a thirteen Gauge needle or any gauge therebetween. Kit 100 also comprises a larger needle 112. Larger needle 112 is typically a gauge larger than guide needle 108, such as a six Gauge needle or a thirteen Gauge needle or any gauge therebetween. Kit 100 also comprises a short cannula 116. Short cannula 116 is typically a 10 Gauge cannula or a fifteen gauge cannula or any gauge therebetween. Kit 100 also comprises a stiff guide wire 120, such as a twenty to twenty-five cm long Amplatz or Rosen wire, or cross wire. Guide wire 120 can be about 0.038 inches to about 0.014 inches in diameter. Kit 100 also comprises a curved needle 124. Needle 124 is typically a one gauge needle or a fifteen gauge needle or any gauge therebetween. Needle 124 can be made from any desired material, but is presently preferred to made from Nitinol. The curve of the needle 124 is chosen to be short and relatively moderate in angulation, so that it will rotate within the confines of the distal radius 32. Diameters for the curve of needle 124 can be the range one mm to one cm, but more particularly in the range of five mm are presently preferred. Cannula 116 can be of a length in the range of about 1-15 cm; 2-13 cm; 3-12cm; 4-11 cm; 5-10 cm; 6-9 cm or 7-8 cm. The gauge of cannula 116 is chosen to allow a working channel exist in the center of the cannula 116 for introduction of other devices. All of the foregoing can be obtained from Cook Group Incorporated (or one of its subsidiaries), P.O. Box 489, Bloomington, Ind. 47402-0489 USA.

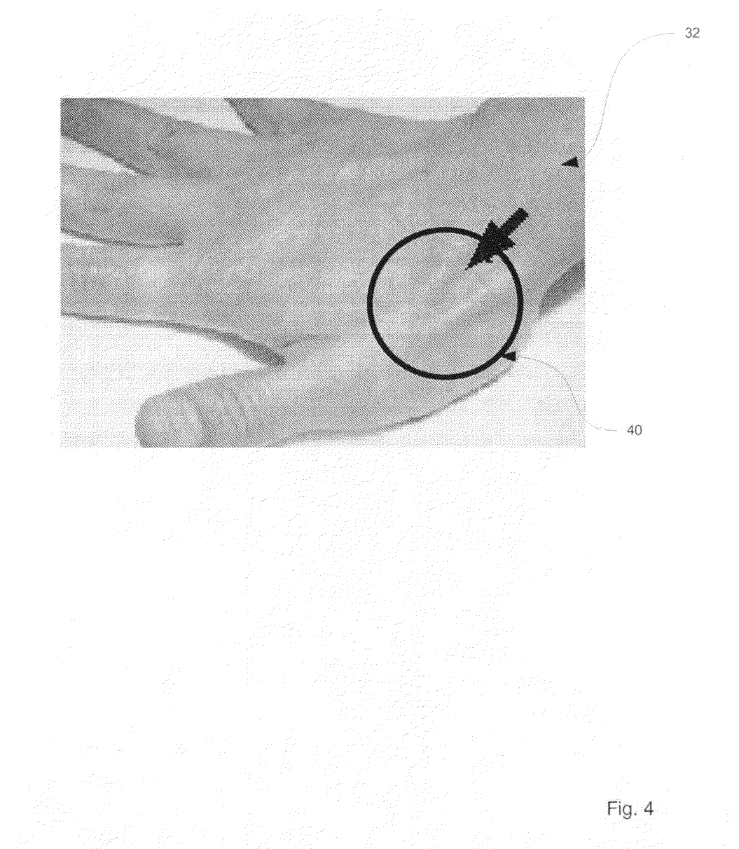

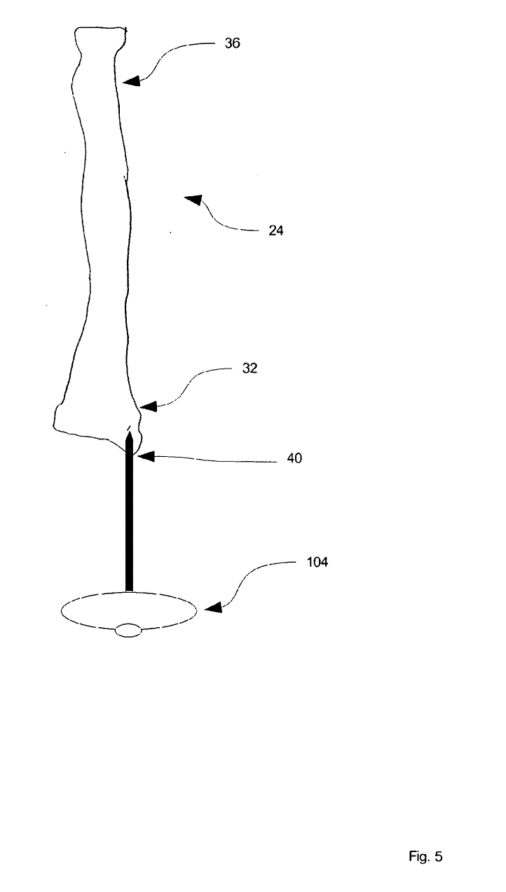

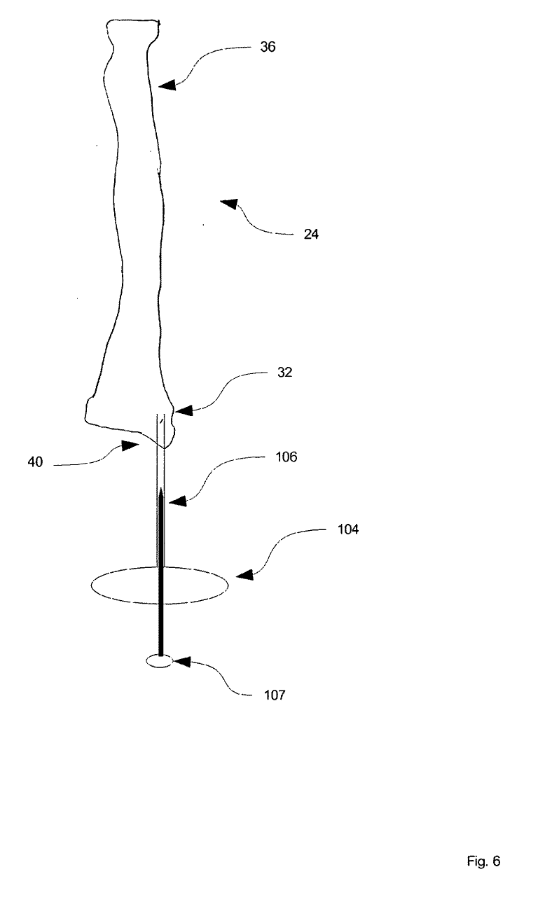

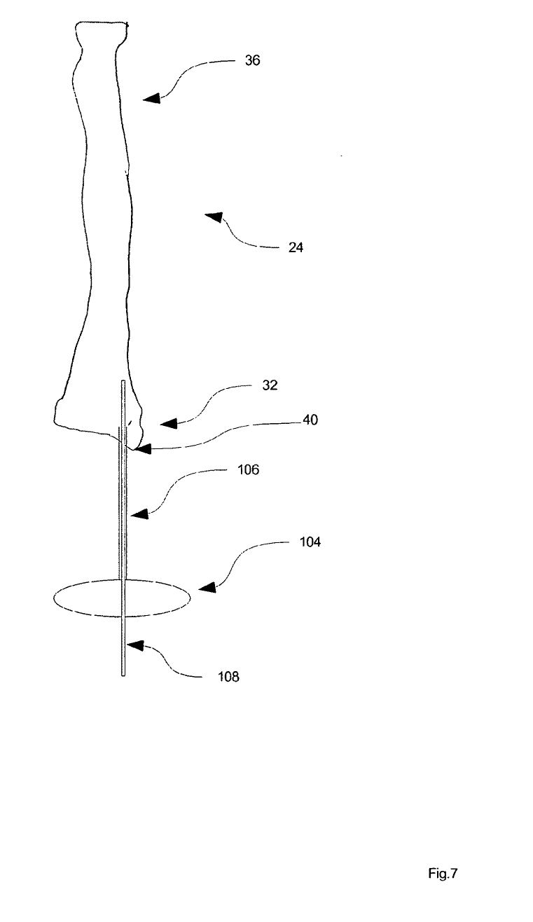

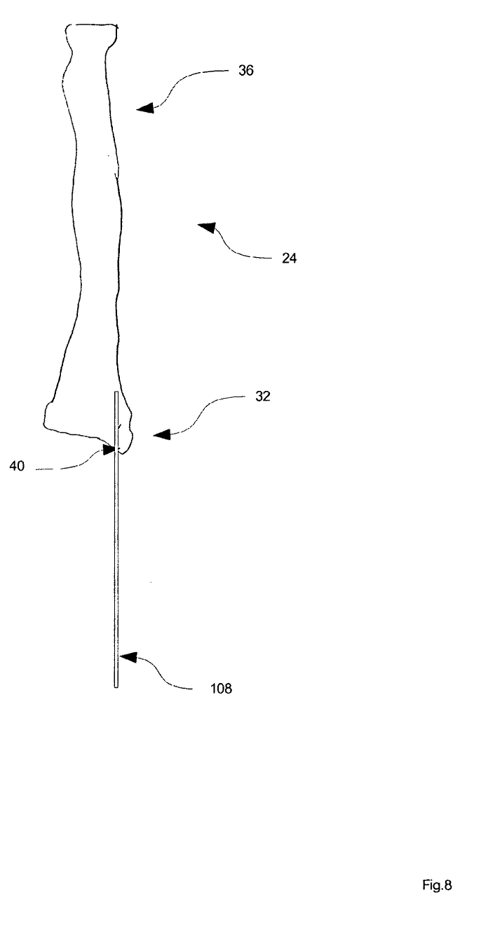

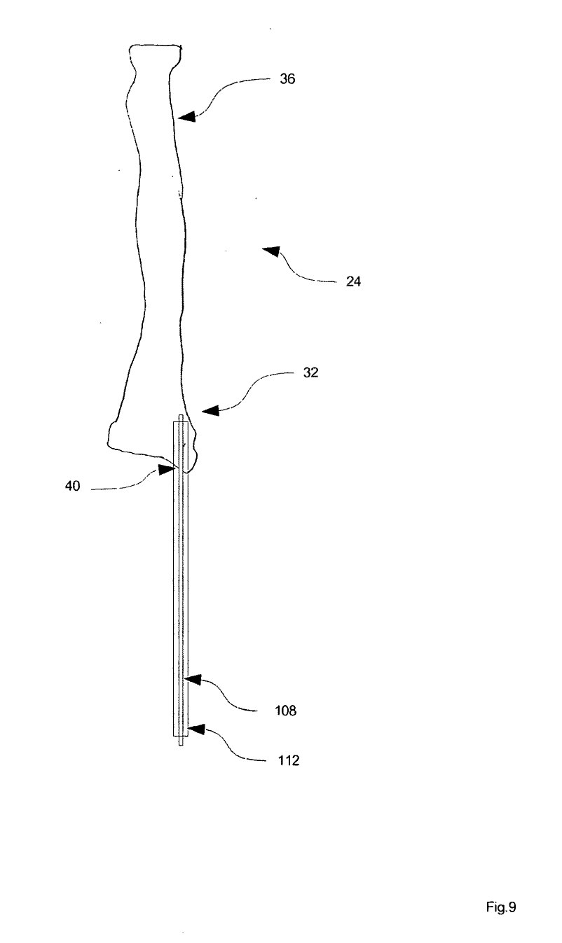

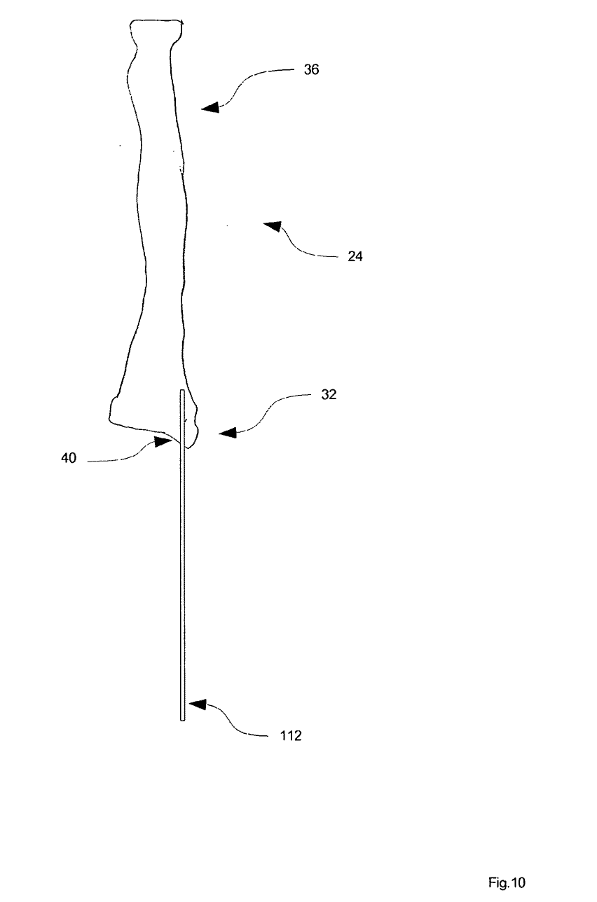

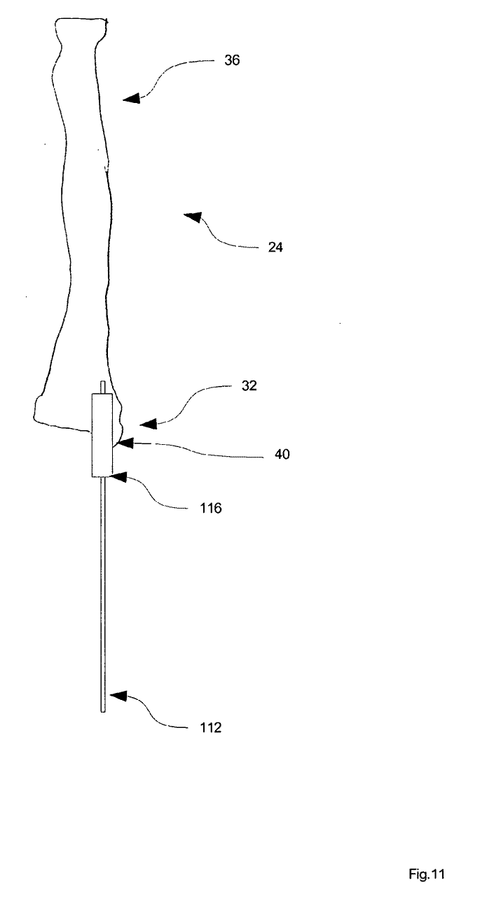

The use of kit 100 to perform the method will now be explained. Referring first to FIG. 4, access is gained to distal radius under 32 fluoroscopic guidance and palpation of boney land marks. Just proximal to the anatomic snuff box 40 of the wrist, under fluoroscopic guidance, and after deliberate avoidance of the basilic vein and the radial hone, a trocar 104, in its assembled form is passed into the radial styloid process 40. FIG. 5 shows a representation of such an entry, but it is to be emphasized that it is a representation presented in a simplified form for the purpose of explaining the present embodiment. Next, as shown in FIG. 6 stylet 107 is removed from cannula 106 leaving a passageway into the interior or radius 24. Through cannula 106, guide needle 108 is inserted, as shown in FIG. 7. Cannula 106 is then removed from radius 24, as shown in FIG. 8. Next, as shown in FIG. 9 a larger needle 112 is inserted over guide needle 108. As shown in FIG. 10, guide needle 108 can then be removed. Larger needle 112 is worked and moved within the opening created in styloid process in such a manner so as to widen the opening and create a path within radius 24 to allow the entry of short cannula 116, as shown in FIG. 11.

Once short cannula 116 is introduced, a stiff guide wire 120 is introduced through cannula 116. Over guide wire 120, a curved needle 124 is then used which can be used to break residual trabecular septations within distal radius 32. This is done by rotating the hub 128 of the curved needle 124, such that the distal end of the curved needle rotates within the distal radius 32 and thereby breaks residual trabecular septations. As will be discussed in greater detail below, trabecular septations can be broken using other instruments.

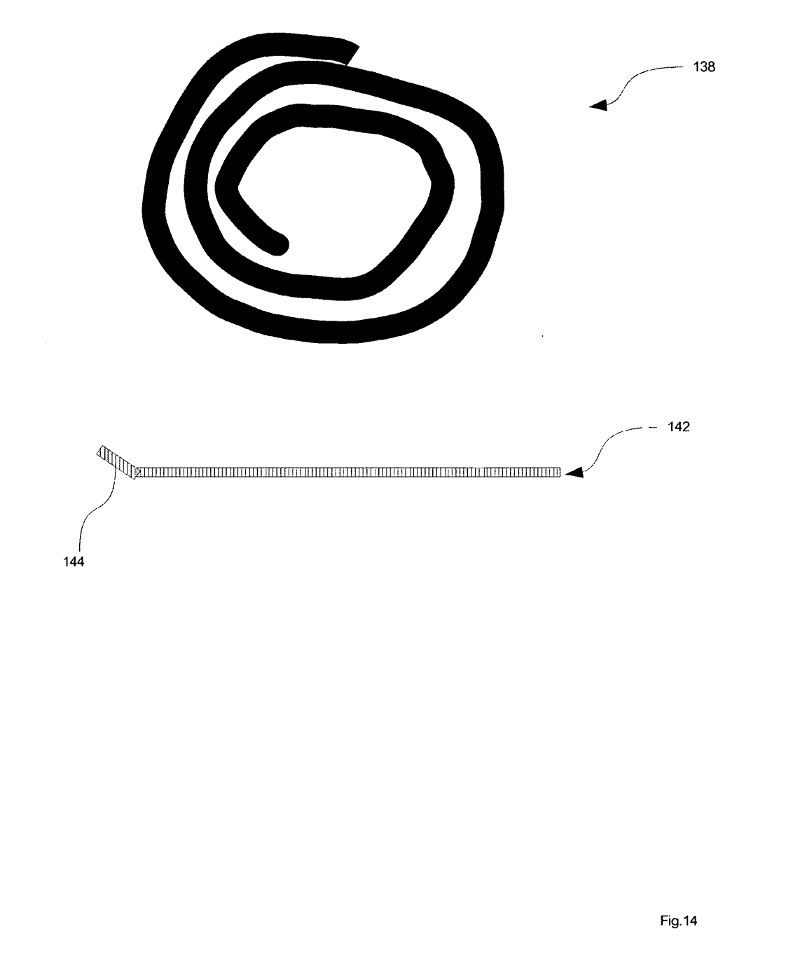

Indeed, additional optional items can be included in kit 100, including a short flexible tube 138 with metal braid as shown in FIG. 14. Where tube 138 is used, then a modified version of guide wire 120, shown as guide wire 142, can be configured to be complementary thereto and each are substantially the same length. Of note, guide 142 includes a bend 144 on its tip. Where tube 138 and guide wire 142 arc provided together, then guide wire 142 can preferably be an Amplatz Super Stiff or Rosen wire available from Cook Group Incorporated (or one of its subsidiaries), P.O. Box 489, Bloomington, Ind. 47402-0489 USA. Tube 138 and/or wire 142 can each be used for, or for assisting in, destruction of the trabecular within the distal radius 32 instead of needle 124. The construction and rigidity of the metal braided tube 138 can be substantially similar to that of a cardiac guided catheter proximally, but modified from cardiac guided catheter in that it is configured to have compressive longitudinal strength of a steel needle.

Also of note, tube 138 can be provided with a dilator at its distal end (not shown) to facilitate its introduction into radius 24. Tube 138 can also be provided with a flexible tip to allow it to be bent into shapes, such as a shape similar to bend 144.

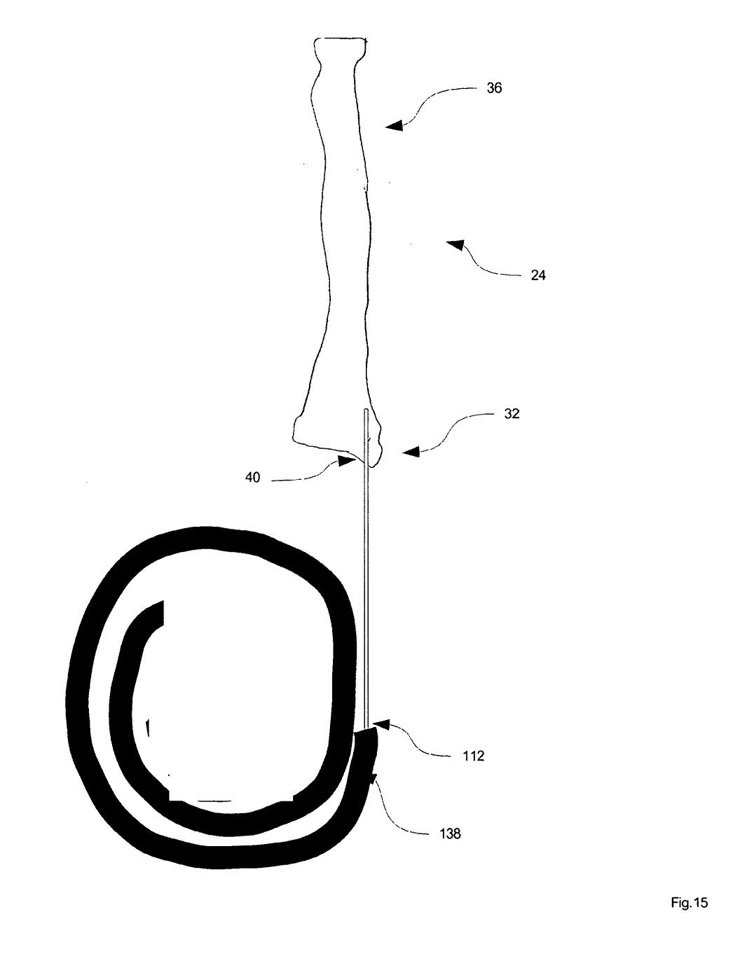

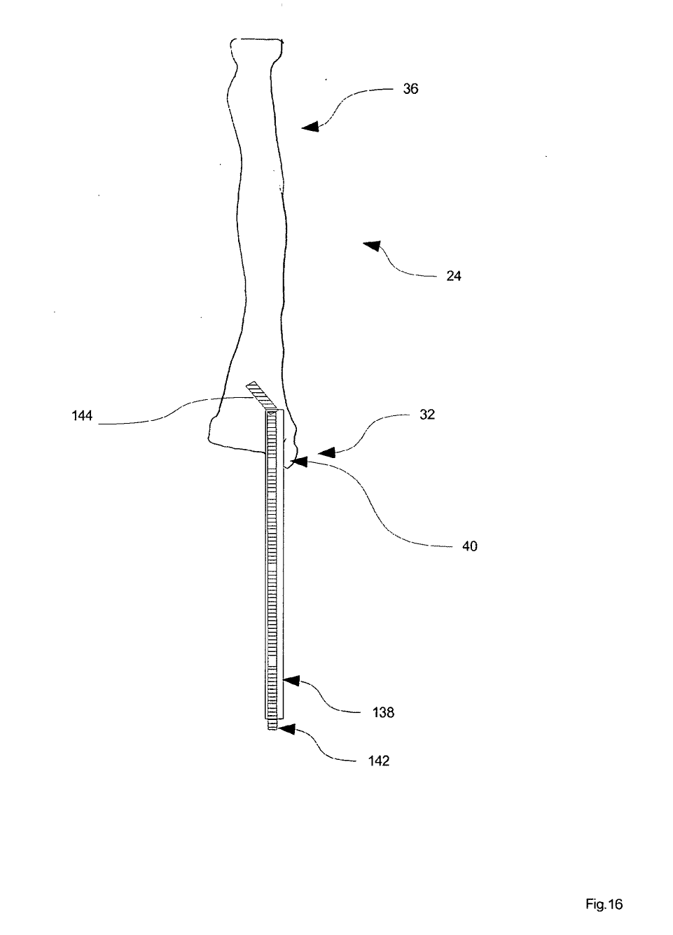

In this variation, the method is performed substantially the same as above having regard to the description accompanying FIGS. 5-10. However, in this embodiment as shown in FIG. 15, tube 138 can be introduced over needle 112 until the distal tip of tube 138 is inside radius 24. The rigidity of tube 138 thus permits tube 138 to be used, if desired, to break residual trabecular septations within radius 24. As shown in FIG. 16, guide wire 142 can be exchanged with needle 112 so that bend 144 of guide wire 142 protrudes from the tip of tube 138. The bend 144 can be used to break residual trabecular septations within radius 24.

As shown in FIG. 16, once a sufficient cavity has been created in radius 24 at a desired target location, a syringe 150 or other dispenser can be connected to tube 138. (In this case, it is preferable that tube 138 include a fitting 154, such as a luer lock, which can be used to connect syringe 150 to fitting 154. Syringe 150 can be filled with a bone cement, such a biologically active bone cement, which can be injected into radius 24. In a present embodiment, metal braided tube 138 has a length sufficient to allow the physician to remove his hands from an x-ray beam that is used to monitor the injection of cement into radius 24. At the same time, tube 138 is also suitable for allowing the injection of bone cement.)

As discussed above, fitting 154, can also be provided at the proximal end of the metal braided tube 138 and is presently preferred. An example of such a fitting can be a luer lock attachment. However, other types of fittings are contemplated, other than luer locks. Whichever type of fitting is chosen, it is presently preferred to select a fitting that can allow injection of cement without increased resistance through the luer assembly. The bone cement can be injected after creation of a cavity in distal radius 32 using the above described method. The bone cement can be injected through needle 124 or short cannula 116, under fluoroscopic guidance. The delivery system can be advanced in the bone to insure that a tract exists and then retracted slightly during the filling process.

Once a pathway is provided between into the interior of distal radius 32, an injection can then be performed. Such an injection can be made through the short flexible tube 138 with fitting 154, such as a luer lock. A suitable bone cement can be delivered through this path. This retraction allows for a path to be created for delivery of cement and decreases pressure at the end of tube 138. Otherwise, the injection requires higher force as the end of the cannula may be against a high resistance object and injection can only occur by reflux of cement along the cannula path. Once the cement is delivered, any items in kit 100 that have been used can be removed. Compression is held over the puncture site to control bleeding. X-ray images can be acquired to verify that alignment of radius 24 is appropriate. The application of image-guided therapy principals and prophylactic cement delivery to patients at high risk is likely to significantly reduce potential morbidity and mortality.

Once the cement has been delivered, it can be desirable to confirm that the cement integrates into the radius 24. The augmentation of the radius 24 response to the bone cement can be heightened by the deliberate delivery of medications, be they delivered orally, subcutaneously, anally or in any other desired manner. Indeed, the bone cement can be made more integrateable within radius 24 by the addition to it of cofactors that stimulate the cement and adjacent boney environment. Therefore, for example and specifically, parathyroid hormone (“PTH”) delivered subcutaneously or orally in the setting of bone cement which may contain a factor such a insulin related growth factor (“IGF”) or Somatomedian is more likely to integrate the bone cement with the surrounding natural bone. Various types of suitable parathyroid hormones are described in US Patent Publication 20060089723, the contents of which are incorporated herein by reference. Having successfully integrated the bone cement and stimulated bone by the use of PTH for periods of time up to one or two years, the patient may then be converted to the use of bone density such as an Editronate diphosphonate drug that can be used to maintain the higher bone density.

While the foregoing describes certain embodiments, it will be understood that combinations, variations, and subsets of those embodiments are contemplated. For example, while the embodiments herein specifically discuss radius 24, other long bones can also be treated using the teachings herein. It should also be understood that the teachings herein can be used to reach any desired target area within a long bone, such as radius 24, and not just the target area shown in the Figures.

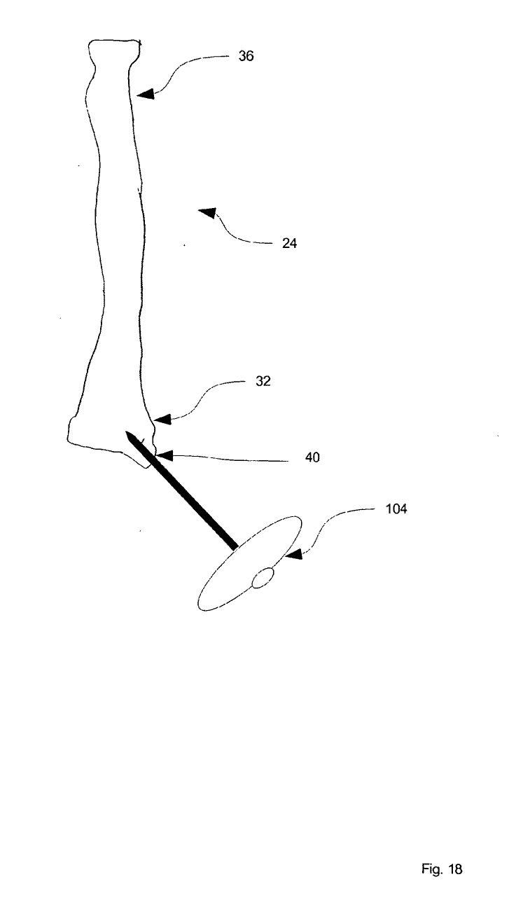

It should be understood that the various Figures are not intended to be “to scale”, and are for representative purposes. Other trajectories and target areas within long bones arc contemplated. For example, in FIG. 18 a diagonal entry through styloid process 40 is shown, and can be presently preferred.

Furthermore, it should be understood that the teachings herein provide certain presently preferred embodiments for creating a channel in a radius through which to inject a bone cement.

However, it should be understood that the embodiments can be modified in order to permit such injection in different ways. For example, once long needle 112 has been introduced in FIG. 10, there are various options for those skilled in the art to employ in order to make use of the conduit presented into the interior of radius 24.

The embodiments herein are intended to be exemplary and the scope of the present invention is defined solely by the claims attached hereto.

1. A method for augmentation of a bone comprising the steps of:
piercing an end of said bone with a trocar until a distal tip of said trocar reaches a target area within said bone, said trocar comprising a stylet and a first cannula;
removing a stylet from said first cannula leaving a passageway through said first cannula into an interior of said bone terminating at said target area;
inserting a guide needle into said first cannula;
passing a larger needle over said guide needle and into said bone;
working said larger needle so as to increase a size of said passageway until said passageway is of a sufficient diameter to receive a second cannula wider than said first cannula; and
inserting said second cannula into said passageway.

2. The method of claim 1, further comprising the step of inserting an instrument into said passageway through said second cannula to break trabecular septations.

3. The method of claim 1, further comprising the steps of:
introducing a stiff guidewire into said second cannula;
passing a hollow curved needle over said stiff guide wire, said hollow curved needle having a curved distal tip, said curve being positioned within said target area; and
rotating a hub of said hollow curved needle such that said curve breaks residual trabecular septations.

4. The method of claim 1, further comprising the step of passing a stiff guide wire into said passageway to breaks residual trabecular septations.

5. The method of claim 4, wherein said stiff guide wire is an Amplatz Super Stiff wire or a Rosen wire.

6. The method of claim 1, further comprising the step of passing a short flexible tube with metal braid into said passageway.

7. The method of claim 6, wherein said short flexible tube is inserted in such a manner as to further break residual trabecular septations.

8. The method of claim 6, wherein said short flexible tube further comprises a fitting for attaching a syringe.

9. The method of claim 8, wherein said syringe is operable to deliver bone cement.

10. The method of claim 6, further comprising the step of injecting bone cement through said flexible tube and into said bone.

11. The method of claim 1, further comprising the step of injecting bone cement into said target area.

12. The method of claim 11, wherein said bone cement is a biologically active bone cement.

13. The method of claim 12, further comprising the step of administering a medication to stimulate said bone cement to integrate with said bone.

14. The method of claim 13, wherein said medication is parathyroid hormone (“PTH”).

15. The method of claim 1, wherein said bone is any one of the distal radius, the proximal femur, the distal and proximal tibia, the proximal femur and the iliac and pelvic bones, the radius, and the humerus.

16. The method of claim 1, wherein said bone has been set after being fractured prior to performance of said method.

17. The method of claim 1, wherein said bone has not been fractured and said method is performed as a prophylactic to reduce the likelihood of fracture of said bone.

18. The method of claim 1, wherein said bone is a vertebral body.

19. The method of claim 1, wherein said vertebral body is T8 and L2.

20. The method of claim 1, wherein said method is performed under image guidance.

21. A method for augmentation of a bone, the method comprising the steps of:
creating a passageway in an end of said bone;
inserting an object into said passageway to break residual trabecula within said bone; and
injecting a biologically active bone cement into said passageway.

22. The method of claim 21, further comprising the step of administering parathyroid hormone (“PTH”) to stimulate said bone cement to integrate with said bone.

23. A kit of parts for augmentation of a bone, the kit comprising:
a trocar comprising a first cannula and a removable stylet, the trocar forming a contiguous tip-for piercing through an end of said bone when said stylet is assembled with said first cannula;
a guide needle for passing through said first cannula into said end of said bone after the end of said bone is pierced by the contiguous tip and said stylet is removed from said first cannula;
a hollow larger needle having a gauge larger than said guide needle, for passing over said guide needle after said first cannula is removed from said end of said bone and while said guide needle is left within said end of said bone, said hollow larger needle is used to break trebuclar septations within said end of said bone to create a passageway of a predefined diameter;
a second cannula for inserting into said passageway of said predefined diameter, the second cannula being adapted such that a first portion of its length inside the bone upon such insertion is substantially equal to a second portion of its length located outside the bone.

24. The kit of claim 23, wherein said second cannula has a length in a range of 8 to 15 cm and a gauge larger than said hollow larger needle and a gauge less than or equal to said predefined diameter.

25. The kit of claim 23, further comprising a stiff guide wire adapted for insertion into said second cannula for breaking residual trabecular septations within said bone.

26. The kit of claim 25, wherein said stiff guide wire is one of an Amplatz wire, a Rosen wire, or a cross wire.

27. The kit of claim 23, further comprising a curved needle, said curved needle having a straight portion and a curved portion at a distal end, said curved portion configured to have a diameter in the range about one mm to about one cm, said curved portion being adapted for breaking residual trabecular septations within said bone when said curved needle is rotated within said bone.

28. The kit of claim 27, wherein said curved portion is configured to have a diameter in the range of about five mm.

29. The kit of claim 23, further comprising a stiff guide wire adapted for insertion into said hollow larger needle and for further optionally breaking said residual trabecular septations.

30. The kit of claim 29, further comprising a metal braided tube passing over said guide wire once said larger needle is removed, said metal tube being adapted for further optionally breaking said residual trabecular septations.
